(19) United States
(12) Patent Application Publication
(10) Pub. No.: US 2025/0244424 A1
(43) Pub. Date: Jul. 31, 2025

(54) TEMPERATURE LIMITING FIXED CURRENT RAMP LEAD FOR SEALED LOW CRYOGEN SUPERCONDUCTING MACHINE

(71) Applicant: GE Precision Healthcare LLC, Waukesha, WI (US)

(72) Inventors: Dogan Celik, Florence, SC (US); Charles Dudley Yarborough, Florence, SC (US); Stuart Paul Feltham, Florence, SC (US); John Edward Book, SR., Pamplico, SC (US)

(21) Appl. No.: 18/428,677

(22) Filed: Jan. 31, 2024

(51) Int. Cl. G01R 33/3815; G01R 33/38; H01F 6/04; H01F 6/06

(52) U.S. Cl. CPC G01R 33/3815; G01R 33/3804; H01F 6/04; H01F 6/065

(57) ABSTRACT

A superconducting machine system includes a superconducting electrical machine and a cryogenic vessel encompassing the superconducting electrical machine. The superconducting machine system includes a ramp lead assembly disposed within a vacuum vessel wall and having a first end and a second end. The first end of the ramp lead assembly is coupled in a fixed manner to the vacuum vessel wall and the second end is coupled to a high temperature superconductor power lead coupled to the superconducting switch. The ramp lead assembly includes a non-conductive support and a metal rod. The ramp lead assembly includes a thermal storage device coupled to the metal rod. The thermal storage device is configured to store heat, to limit heat transfer along the metal rod, and to limit an increase in temperature along the ramp lead assembly during the energization of the superconducting electrical machine.

### BACKGROUND

The subject matter disclosed herein relates to a temperature limiting fixed current ramp lead for sealed low cryogen superconducting machine.

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During magnetic resonance imaging (MRI), when a substance such as human tissue is subjected to a uniform magnetic field (polarizing field B0), the individual magnetic moments of the spins in the tissue attempt to align with this polarizing field, but precess about it in random order at their characteristic Larmor frequency. If the substance, or tissue, is subjected to a magnetic field (excitation field B1) which is in the x-y plane and which is near the Larmor frequency, the net aligned moment, or “longitudinal magnetization”, M_{z}, may be rotated, or “tipped”, into the x-y plane to produce a net transverse magnetic moment, Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

MRI systems may utilize superconducting magnets using NbTi conductor, other superconductors can be employed at similar or higher temperatures to NbTi. Also, MRI systems require a cooling system may utilize a cryogen (very cold liquid) to operate but magnets can be directly cooled leveraging conductive materials (such as copper). Ramp lead are utilized for energizing superconducting MRI magnets (as well as other types of superconducting machines such as motors and generators). Often times, these ramp leads can be fixed (permanently in place) or detachable/retractable (either method looks to break the electrical and thermal link between room temperature and the magnet and, thus, have moving parts. Ramp leads that are not completely removed after use will cause a parasitic heat leak in the superconducting MRI magnet.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a superconducting machine system is provided. The superconducting machine system includes a superconducting electrical machine. The superconducting machine system also includes a cryogenic vessel. The cryogenic vessel encompasses the superconducting electrical machine. A vacuum vessel wall encompasses the cryogenic vessel. The superconducting machine system also includes a superconducting switch coupled to the superconducting electrical machine and configured to switch between a resistive mode and a superconducting mode. The superconducting machine system further includes a ramp lead assembly disposed within the vacuum vessel wall and having a first end and a second end. The first end of the ramp lead assembly is coupled in a fixed manner to the vacuum vessel wall and the second end is coupled to a high temperature superconductor power lead coupled to the superconducting switch. The ramp lead assembly includes a non-conductive support. The ramp lead assembly also includes a metal rod configured to minimize static heat load, wherein the metal rod is disposed on the non-conductive support and extends between the first end and the second end. The ramp lead assembly further includes a thermal storage device coupled to the metal rod between the first end and the second end. The thermal storage device is configured to store heat during energization of the superconducting electrical machine, to limit heat transfer along the metal rod to the second end during the energization of the superconducting electrical machine, and to limit an increase in temperature along the ramp lead assembly during the energization of the superconducting electrical machine. A high temperature superconductor portion of the high temperature superconductor power lead is disposed between the second end of the metal rod and the superconducting switch.

In another embodiment, a superconducting magnet system for a magnetic resonance imaging system is provided. The superconducting magnet system includes a superconducting magnet. The superconducting magnet system also includes a cryogenic vessel encompassing the superconducting magnet. A vacuum vessel wall encompasses the cryogenic vessel The superconducting magnet system further includes a superconducting switch coupled to the superconducting magnet and configured to switch between a resistive mode and a superconducting mode. The superconducting magnet system even further includes a ramp lead assembly disposed within the vacuum vessel wall and having a first end and a second end, wherein the first end of the ramp lead assembly is coupled in a fixed manner to the vacuum vessel wall and the second end is coupled to a high temperature superconductor power lead coupled to the superconducting switch. The ramp lead assembly includes a non-conductive support. The ramp lead assembly also includes a metal rod configured to minimize static heat load, wherein the metal rod is disposed on the non-conductive support and extends between the first end and the second end. The ramp lead assembly further includes a solid metal structure coupled to the metal rod between the first end and the second end, wherein the ramp lead assembly is configured so that heat, during energization of the superconducting magnet, preferentially flows to the solid metal structure instead of along the metal rod toward the second end. A high temperature superconductor portion of the high temperature superconductor power lead is disposed between the second end of the metal rod and the superconducting switch.

In a further embodiment, a method for limiting temperature of a ramp lead assembly coupled to a superconducting magnet for a magnetic resonance imaging system is provided. The method includes energizing the superconducting magnet disposed within a cryogenic vessel disposed within a vacuum vessel wall. The magnetic resonance imaging system includes a ramp lead assembly disposed within the vacuum vessel wall and having a first end and a second end, wherein the first end of the ramp lead assembly is coupled in a fixed manner to the vacuum vessel wall and the second end is coupled to a high temperature superconductor power lead coupled to a superconducting switch. The ramp lead assembly includes a non-conductive support. The ramp lead assembly also includes a metal rod that minimize static heat load during energization of the superconducting magnet, wherein the metal rod is disposed on the non-conductive support and extends between the first end and the second end. A high temperature superconductor portion of the high temperature superconductor power lead is disposed between the second end of the metal rod and the superconducting switch that is configured to further reduce heat transfer into the superconducting magnet. The ramp lead assembly further includes a thermal storage device coupled to the metal rod between the first end and the second end. The method also includes storing heat during energization of the superconducting magnet, limiting heat transfer along the metal rod to the second end during the energization of the superconducting magnet, and limiting an increase in temperature along the ramp lead assembly during the energization of the superconducting magnet.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a schematic diagram of an example magnetic resonance system, in accordance with aspects of the present disclosure;

FIG. 2 is a schematic diagram of a current or power ramp lead coupled to a superconducting switch, in accordance with aspects of the present disclosure;

FIG. 3 is a schematic diagram of a current ramp lead for utilization with a superconducting magnet of the magnetic resonance system in FIG. 1, in accordance with aspects of the present disclosure;

FIG. 4 is a schematic diagram of a current ramp lead (e.g., having a thermal storage device) for utilization with a superconducting magnet of the magnetic resonance system in FIG. 1, in accordance with aspects of the present disclosure;

FIG. 5 is a perspective view of a fixed ramp lead assembly for a pair of current ramp leads, in accordance with aspects of the present disclosure;

FIG. 6 is a perspective view of the fixed ramp lead assembly in FIG. 5, in accordance with aspects of the present disclosure;

FIG. 7 is a top view of the fixed ramp lead assembly in FIG. 5, in accordance with aspects of the present disclosure;

FIG. 8 is a schematic view of a baseline fixed ramp lead assembly, in accordance with aspects of the present disclosure;

FIG. 9 is a schematic view of an improved revision fixed ramp lead assembly (e.g., having a thermal storage device), in accordance with aspects of the present disclosure;

FIG. 10 is a graph illustrating the heat going into a thermal anchor during a simulated ramping process, in accordance with aspects of the present disclosure;

FIG. 11 is an end view and a side view of a thermal storage device (e.g., simple disc), in accordance with aspects of the present disclosure;

FIG. 12 is an end view of a thermal storage device (e.g., disc with tapered bore), in accordance with aspects of the present disclosure;

FIG. 13 is an end view of a thermal storage device (e.g., disc with tapered bore and additional material), in accordance with aspects of the present disclosure;

FIG. 14 is an end view of a thermal storage device (e.g., disc with hybrid materials), in accordance with aspects of the present disclosure;

FIG. 15 is schematic view of a thermal storage device (e.g., cryogen tank), in accordance with aspects of the present disclosure; and

FIG. 16 is a schematic diagram of a current or power ramp lead coupled to a superconducting switch coupled to a superconducting electrical machine, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as power generation using superconducting machines. In general, the technique can be used in all superconducting machinery requiring energy transfer to/from a much higher temperature environment.

The present disclosure provides a system and a method for a current ramp lead assembly to limit a temperature of a superconducting electrical machine (e.g., superconducting magnet of a magnetic resonance imaging system, motor, generator, etc.) in a sealed or bath (e.g., holding liquid cryogen such liquid helium) cryogenic system during energization of the superconducting electrical machine. In particular, a superconducting machine system includes a superconducting electrical machine. The superconducting machine system can also include a sealed cryogenic (e.g., helium) vessel encompassing the superconducting electrical machine. The superconducting machine system also includes a vacuum vessel wall encompassing the sealed cryogenic vessel. The superconducting machine system also includes a superconducting switch coupled to the superconducting electrical machine and configured to switch between a resistive mode and a superconducting mode. The superconducting machine system further includes a ramp lead assembly disposed within the sealed cryogenic vessel and having a first end and a second end. The first end of the ramp lead assembly is coupled in a fixed manner to the vacuum vessel wall and the second end is coupled to a high temperature superconductor power lead coupled to the superconducting switch. The ramp lead assembly includes a non-conductive support. The ramp lead assembly also includes a metal rod configured to minimize static heat load, wherein the metal rod is disposed on the non-conductive support and extends between the first end and the second end. The ramp lead assembly further includes a thermal storage device (e.g., thermal battery) coupled to the metal rod between the first end and the second end. The thermal storage device is configured to store heat during energization of the superconducting electrical machine, to limit heat transfer along the metal rod to the second end during the energization of the superconducting electrical machine, and to limit an increase in temperature along the ramp lead assembly during the energization of the superconducting electrical machine.

In certain embodiments, the first end and the second end of the fixed ramp lead assembly are both are made of copper and the metal rod is made of a relatively lower thermally conductive metal such as brass. In certain embodiments, the first end and the second of the fixed ramp lead assembly are both made of brass. In certain embodiments, a portion of the first end is located outside the vacuum vessel wall, wherein a temperature outside the vacuum vessel wall is an ambient temperature. In certain embodiments, the second end is coupled to a thermal anchor (e.g., a thermal shield that the sealed cryogenic vessel is located within).

In certain embodiments, the thermal storage device is configured to limit the heat flowing into the thermal anchor during the energization of the superconducting electrical machine. In certain embodiments, the ramp lead assembly is configured so that the heat during the energization of the superconducting electrical machine preferentially flows to the thermal storage device instead of along the metal rod toward the second end.

In certain embodiments, the thermal storage device includes a cryogen tank coupled to the metal rod. In certain embodiments, the thermal storage device is solid structure made of a solid material with a high heat capacity (e.g., material with heat capacities greater than 250 Joules per kilogram per Kelvin (J/kg-K) at 100 K temperature) that limits a temperature increase by absorbing heat. In certain embodiments, the thermal storage device is made of metal. In certain embodiments, the thermal storage device is a metal disc disposed about the metal rod at a location between the first end and the second end of fixed ramp assembly. In certain embodiments, the metal disc is made of brass or copper. In certain embodiments, the thermal storage device includes a plurality of metal (e.g., brass or copper) discs disposed about the metal rod spaced apart from each other.

In certain embodiments, one or more of the metal discs of the thermal storage device include a tapered bore that the metal rod passes through, wherein the tapered bore is configured to reduce axial conduction along the metal rod. In certain embodiments, one or more of the metal discs of the thermal storage device include a central portion having the tapered bore and an additional portion disposed along a perimeter of the central portion. The central portion extends in a first direction and the additional portion extends in a second direction crosswise to the first direction. The additional portion is configured to increase a capacity of the respective metal disc to store the heat compared to a metal disc only having the central portion. In certain embodiments, the central portion is made of a first metal, and the additional portion is made of second metal different from the first metal.

In certain embodiments, a superconducting magnet system for a magnetic resonance imaging system includes a superconducting magnet. The superconducting magnet system also includes a sealed cryogenic vessel encompassing the superconducting magnet. The superconducting magnet system also includes a vacuum vessel wall encompassing the sealed cryogenic vessel. The superconducting magnet system further includes a superconducting switch coupled to the superconducting magnet and configured to switch between a resistive mode and a superconducting mode. The superconducting magnet system even further includes a ramp lead assembly disposed within the vacuum vessel wall and having a first end and a second end, wherein the first end of the ramp lead assembly is coupled in a fixed manner to the vacuum vessel wall and the second end is coupled to a high temperature superconductor power lead coupled to the superconducting switch. The ramp lead assembly includes a non-conductive support. The ramp lead assembly also includes a metal rod configured to minimize static heat load, wherein the metal rod is disposed on the non-conductive support and extends between the first end and the second end. The ramp lead assembly further includes a solid metal structure coupled to the metal rod between the first end and the second end, wherein the ramp lead assembly is configured so that heat during the energization of the superconducting magnet preferentially flows to the solid metal structure instead of along the metal rod toward the second end.

In certain embodiments, a method for limiting temperature of a ramp lead assembly coupled to a superconducting magnet for a magnetic resonance imaging system includes energizing the superconducting magnet disposed within a sealed cryogenic vessel disposed within a vacuum vessel wall. The magnetic resonance imaging system includes a ramp lead assembly disposed within the vacuum vessel wall and having a first end and a second end, wherein the first end of the ramp lead assembly is coupled in a fixed manner to the vacuum vessel wall and the second end is coupled to a high temperature superconductor power lead coupled to a superconducting switch. The ramp lead assembly includes a non-conductive support. The ramp lead assembly also includes a metal rod that minimize static heat load during energization of the superconducting magnet, wherein the metal rod is disposed on the non-conductive support and extends between the first end and the second end. The ramp lead assembly further includes a thermal storage device coupled to the metal rod between the first end and the second end. The method also includes storing heat during energization of the superconducting magnet, limiting heat transfer along the metal rod to the second end during the energization of the superconducting magnet, and limiting an increase in temperature along the ramp lead assembly during the energization of the superconducting magnet.

The disclosed embodiments provide for fixed/permanent ramp lead assembly that can be installed on a low cryogen superconducting electrical machine (e.g., superconducting magnet) that provides an always connected electrical path between an external power supply and an internal circuit of the superconducting electrical machine. Thus, the disclosed embodiments are suitable for automatic or remote ramping-up/ramping down of low cryogen sealed superconducting electrical machines that have very limited cryogen (e.g., a few liters of helium) as well as superconducting electrical machines in a traditional liquid cryogen vessel. The disclosed embodiments enables the automatic ramping up/down of a superconducting magnet in case of a power outage or a natural disaster. The disclosed embodiments can increase superconducting magnet availability for scanning by avoiding a quench for low cryogen (as the recovery from a quench can take several days). The disclosed embodiments enable the reduction of heat transfer during operation of the superconducting electrical machine. The disclosed embodiments provides for the flow of thermal energy into a thermal storage device to limit a temperature rise and to limit heat flowing into a thermal anchor during a ramping process. The disclosed embodiments provide for a simple, low cost, and robust configuration for ramp leads that used for energizing superconducting electrical machines.

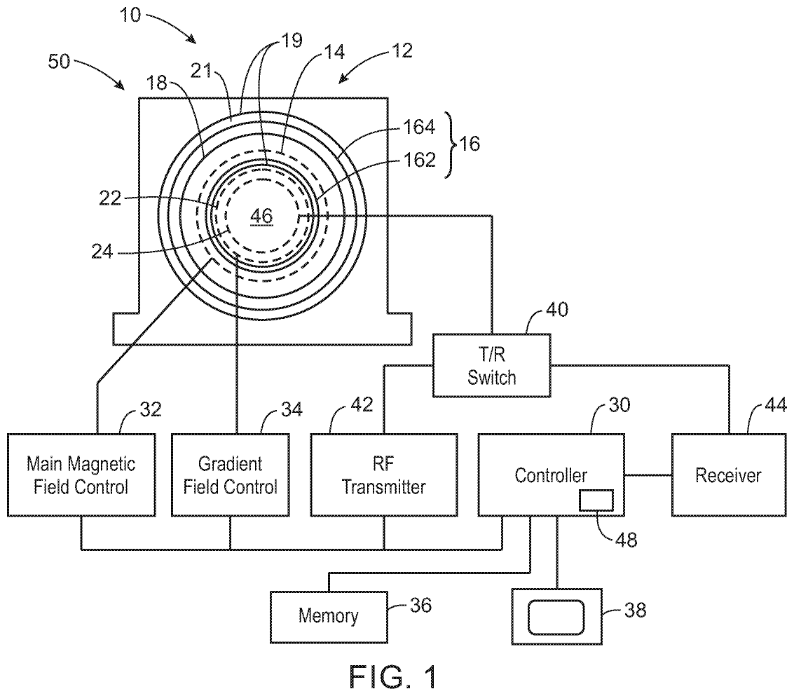

Referring now to FIG. 1, a schematic block diagram of an example MR system 10 is illustrated. MR system 10 is used to obtain images or for spectroscopy applications of a subject. In the example embodiment, MR system 10 includes a magnet assembly 12 that includes a magnet 14. In some embodiments, magnet 14 is a superconducting magnet formed from a plurality of magnetic coils wound around a magnetic coil support or a coil former. Magnet 14 is configured to generate a polarizing magnetic field. As described in greater detail below, the MR system 10 includes a current ramp lead assembly to limit a temperature of a superconducting magnet 14.

Magnet assembly 12 may include a cryostat vessel 18 that surrounds magnet 14. Cryostat vessel 18 is typically filled with a cryogenic fluid or cryogen which is used to cool the superconducting coils into an extremely low temperature, e.g., 4 kelvin (K), such that electric current continues to flow through the superconducting coils without electrical resistance to maintain a uniform and static magnetic field after a power supply is disconnected. Cryogen may be helium, hydrogen, neon, nitrogen, or any combination thereof, in a liquid form, a gaseous form, or a combination of liquid and gaseous form. Helium is described as an example cryogen. Other cryogen may be used in superconducting magnet assemblies described herein.

In the example embodiment, magnet assembly 12 may also include a thermal shield assembly 16 that enclose cryostat vessel 18 and magnet 14 therein. In one embodiment, thermal shield assembly 16 may include an inner thermal shield member 162 and an outer thermal shield member 164. Inner thermal shield member 162 is generally cylindrical in shape and is radially placed inside of magnet 14. Inner thermal shield member 162 is configured to prevent heat being radiated from a warm region where the subject is placed to a cold region where magnet 14 is placed. Outer thermal shield member 164 is arranged concentrically with respect to inner thermal shield member 162. Outer thermal shield member 164 may also have a generally cylindrical shape and is radially placed outside of magnet 14. Outer thermal shield member 164 is configured to prevent heat being radiated from environment into magnet 14. Thermal shield assembly 16 is made from metal materials, such as aluminum. In some embodiments, magnet assembly 12 may also include a magnet vacuum vessel 19 (e.g. having a vacuum vessel wall 21) surrounding thermal shield assembly 16 and insulating magnet 14 from the environment during operation.

In the example embodiment, MR system 10 also includes a gradient coil assembly 22 placed inside of inner thermal shield member 162. Gradient coil assembly 22 is configured to selectively impose one or more gradient magnetic fields along one or more axes, such as x, y, or z axes. MR system 10 also includes RF coil 24. RF coil 24 may be a transmitter coil, which is configured to transmit RF pulses. RF coil 24 may be a receiver coil, which is configured to detect MR signals from the subject. RF coil 24 may be a transmit and receive coil that transmits and also detect MR signals. Magnet assembly 12, gradient coil assembly 22, and body RF coil 24 are collectively referred to as a scanner assembly 50, because scanner assembly 50 forms into one unit and is in a scanner room. Scanner assembly 50 has a bore 46, where the subject is positioned during scanning. Scanner assembly 50 shown in FIG. 1 is a closed bore system, where the bore is cylindrical. Scanner assembly 50 may be magnet assemblies of other designs, such as an open-bore system, a dipolar electromagnet configuration, or a Hallbach configuration.

In the example embodiment, MR system 10 also includes a controller 30, a magnetic field control 32, a gradient field control 34, a memory 36, a display device 38, a transmit/receive (T/R) switch 40, an RF transmitter 42, and a receiver 44. In operation, a subject is placed in bore 46 on a suitable support, for example, a motorized table (not shown) or other patient table. Magnet 14 produces a uniform and static magnetic field B0 across bore 46. Strength and homogeneity of the magnet field B0 in bore 46 and correspondingly in patient is controlled by controller 30 via magnetic field control 32, which also controls a supply of energized current to magnet 14. Gradient coil assembly 22 is energized by gradient field control 34 and is also controlled by controller 30, so that one or more gradient magnetic fields are imposed on the magnetic field B0. RF coil 24 and a receive coil, if provided, are selectively interconnected to one of RF transmitter 42 or receiver 44, respectively, by T/R switch 40. RF transmitter 42 and T/R switch 40 are controlled by controller 30 such that RF field pulses or signals are generated by RF transmitter 42 and are selectively applied to the subject for excitation of magnetic resonance in the subject.

In the example embodiment, following application of the RF pulses, T/R switch 40 is again actuated to decouple RF transmit coil 24 from RF transmitter 42. The detected MR signals are in turn communicated to controller 30 which may organize the MR signals in a particular format for storage in memory 36. Controller 30 includes a processor 48 that controls the processing of the MR signals to produce signals representative of an image of the patient, which are transmitted to display device 38 to provide a visual display of the image.

Magnet 14 of magnet assembly 12 is used to generate a magnetic field in MR system 10 by electric current flowing along magnet windings of magnet 14. The current is in a range of hundreds of amperes. In some known systems, an electric current from a power source is constantly applied to the magnet to produce the magnetic field. A constant supply of the high electric current would significantly increase the running cost of an MR system. Magnet 14 is a superconducting magnet, where magnet 14 operates at a superconducting temperature, such as 4 K, of wire windings of magnet 14 such that winding wires do not have electrical resistance to the current and external power source is not needed. This mode of operation of magnet 14 is referred to as a persistent current mode.

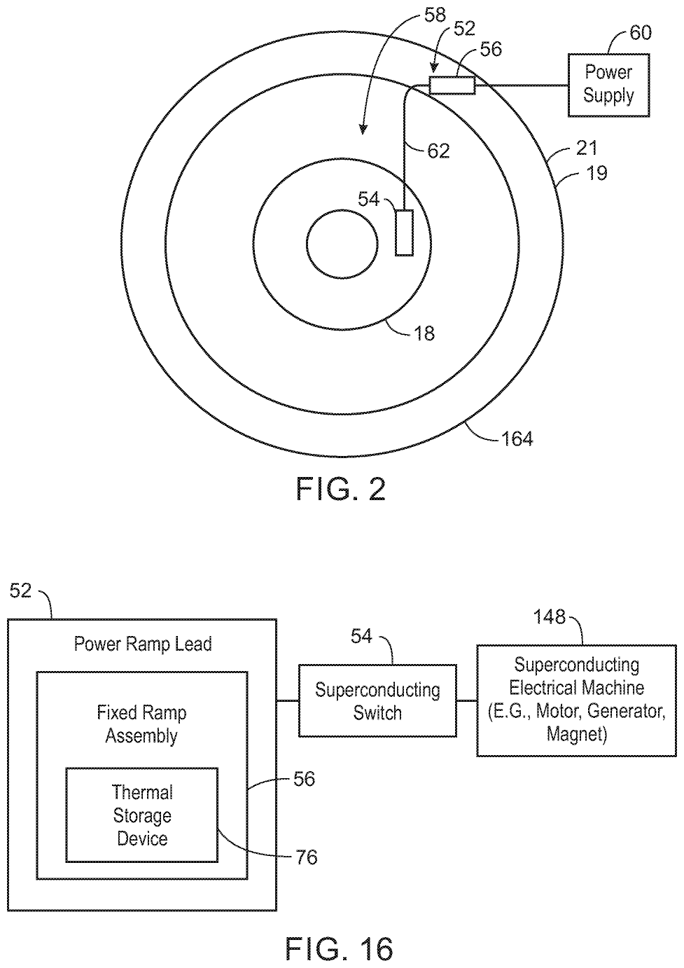

FIG. 2 is a schematic diagram of a current or power ramp lead 52 coupling to a superconducting switch 54. As depicted, the superconducting switch 54 is located within the cryostat (e.g., cryogenic) vessel 18. In certain embodiments, the superconducting switch 54 may be located between outer thermal shield member 164 and the cryogenic vessel 18. The superconducting switch 54 is used to excite the magnet to operate in a persistent current mode. The superconducting switch 54 includes windings. The superconducting switch 54 switches between a resistive mode and a superconducting mode. In the resistive mode, the superconducting switch 54 operates at a temperature above the superconducting temperature of windings of the switch, such as 80 K. In the superconducting mode, the superconducting switch 54 operates at the superconducting temperature, such as 4 K, of the windings of the superconducting switch 54. The superconducting switch 54 is electrically connected the power source in parallel with the magnet, during ramp-up of the magnet. To generate the magnetic field, the superconducting switch 54 operates in the resistive mode such that electric current is injected to the switch due to the resistance in windings of the switch at the resistance mode. When the electric current flowing through the superconducting switch 54 reaches a desired level for producing the field strength of the magnetic field, the external power source is disconnected and the superconducting switch 54 is switched to operate at the superconducting mode. The switching is accomplished by changing the temperature of superconducting switch 54. A heater is used to heat the superconducting switch 54. The superconducting switch 54 is typically cooled by cryogen such as liquid helium. As a result, heating of the superconducting switch 54 boils off the cryogen.

During ramp-down or quench of the magnet, the magnetic field produced by the magnet is reduced by switching the superconducting switch 54 to operate from the superconducting mode to the resistive mode. The flow of current through resistance generates heat, which also boils off the cryogen.

The current or power lead 52 includes a fixed (or permanent) ramp lead assembly 56 disposed within a space 58 between the vacuum vessel wall 21 of the magnet vacuum vessel 19 and the outer thermal shield member 164. A portion of the fixed ramp assembly is located outside the vacuum vessel wall (where the temperature is an ambient temperature) and is coupled to an external power supply 60. The current or power lead 52 also includes a high temperature superconducting power lead 62 that couples to and provides electrical power to the superconducting switch 54.

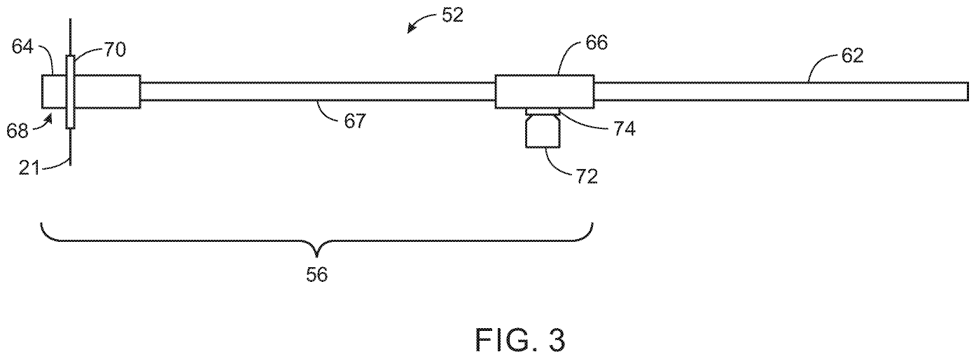

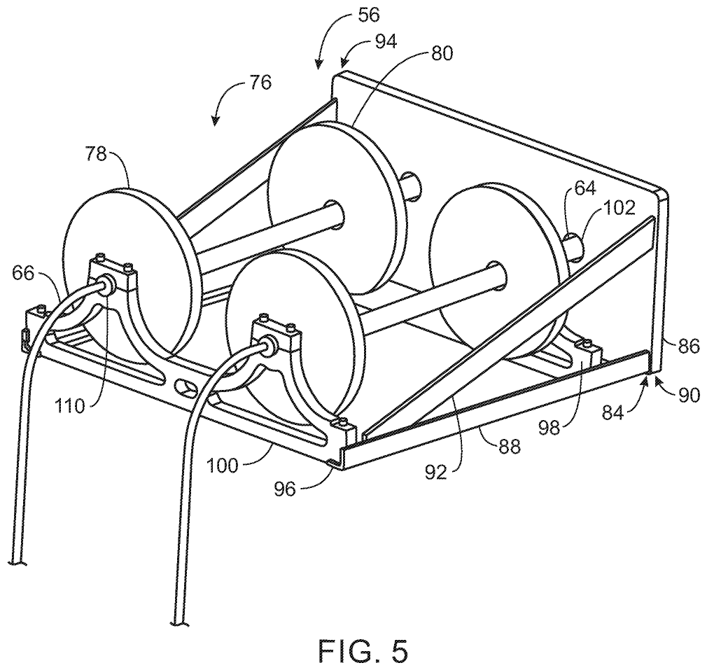

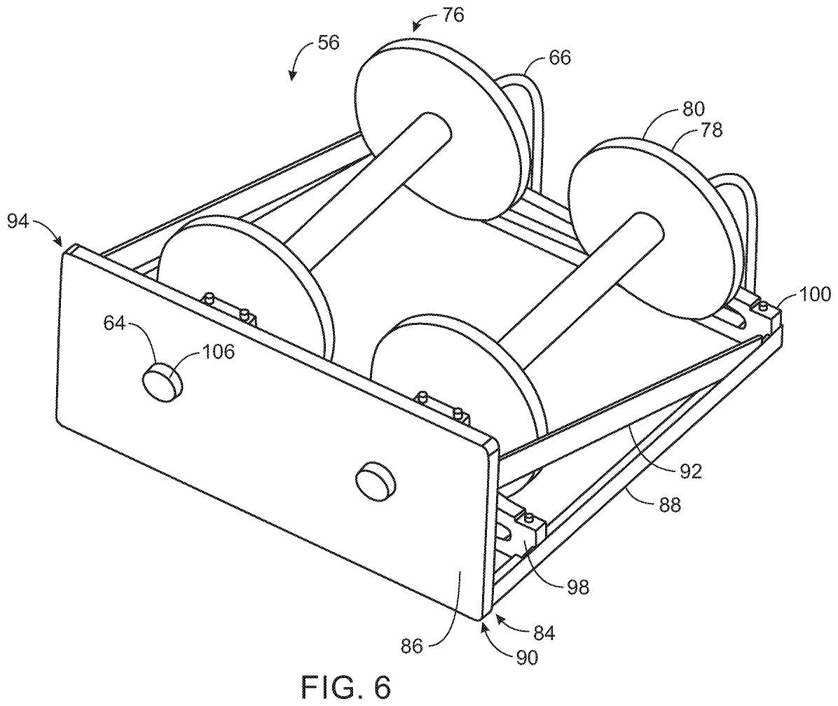

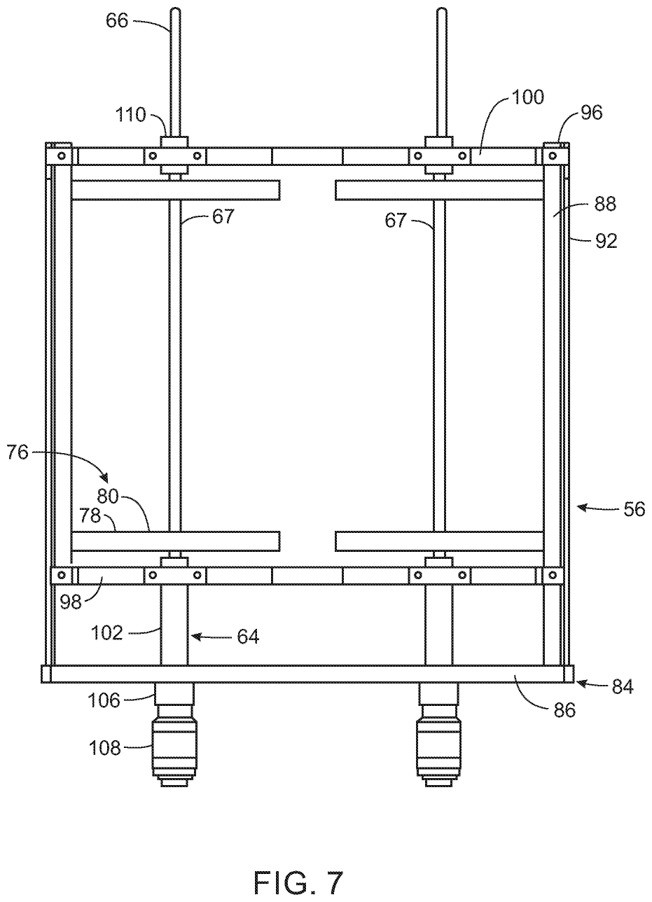

FIG. 3 is a schematic diagram of a current ramp lead 52 for utilization with a superconducting magnet of the magnetic resonance system 10 in FIG. 1. The current ramp lead 52 includes the fixed ramp lead assembly 56 and the high temperature superconducting power lead 62. The high temperature superconducting power lead 62 may include high temperature superconducting tape (e.g., yttrium barium copper oxide or RBCO) disposed a conductive wire. In certain embodiments, the fixed ramp lead assembly 56 includes a non-conductive support (see FIGS. 5-7).

As depicted, the fixed ramp lead assembly 56 includes a first end 64 and a second end 66 (e.g., metal ends). The first end 64 and the second end 66 are made of copper. In certain embodiments, the first end 64 and the second end 66 are made of brass. A metal rod 67 extends between the first end 64 and the second end 66. The metal rod 67 is made of a relatively lower thermal conductive material such as brass. The metal rod 67 is configured to minimize static heat load. During operation (e.g. energization of the superconducting magnet), a temperature gradient is formed along the metal rod 67 (with it being warmer the first end 64 and cooler at the second end 66). The metal rod 67 has a smaller cross-section than the metal ends 64, 66.

The first end 64 is coupled to the vacuum vessel wall 21. The fixed ramp lead assembly 56 is coupled to the vacuum vessel wall 21 in a fixed or permanent manner to enable the current ramp lead 52 to provide an always connected electrical path between the external power supply and the superconducting magnet internal electrical circuit. This eliminates the need for moving parts (e.g., compared to a detachable/retractable ramp lead). A portion 68 of the first end 64 is disposed outside the vacuum vessel wall 21 where the temperature is an ambient temperature. An electrical insulator 70 (e.g., ceramic insulator) is provided where the portion 68 of the first end 64 extends through the vacuum vessel wall 21. The remaining portion of the fixed ramp lead assembly 56 is disposed in the space between the vacuum vessel wall 21 and the thermal shield (e.g., outer thermal shield member 164 in FIG. 1).

The second end 66 is coupled to the high temperature superconducting power lead 62 (e.g., coupled to the high temperature superconducting portion of the high temperature superconducting power lead 62 which is coupled to the superconducting switch). The higher temperature superconducting power lead 62 has a temperature limit to stay superconducting (e.g., below liquid nitrogen temperature, 77K). Therefore, the second end 66 is coupled to a thermal anchor 72 prior to the high temperature superconducting power lead 62. The thermal shield (in particular, the outer thermal shield member 164) in FIGS. 1 and 2 serves as the thermal anchor 72. An electrical insulator 74 (e.g., ceramic insulator) is disposed between the second end 66 and the thermal anchor 72.

A balance between steady/parasitic heat leak from the ambient temperature area to the superconducting magnet occurs in the configuration of the current ramp lead 52 in FIG. 3. Also, joule (e.g., resistive) heat generation needs to be minimized and temperature limited during energization/ramping of the superconducting magnet. If a good conductor with larger cross-section and/or shorter length is utilized to minimize the resistive heat generation during energization of the magnet, then the parasitic heat load to the thermal anchor 72 during normal operation of the superconduct magnet will be too great to handle. Therefore, one has to utilize smaller cross-sections and/or longer lengths of metal conductors (e.g., of the metal rod 67). Alternatively, lower thermal conductive material such as brass may be utilized. However, any material with proper thermal and electrical properties can be utilized. However, any attempt to lower permanent/parasitic head load will end up creating a larger resistive heating which will flow to the thermal anchor 72 and increase the overall temperature in the current ramp lead 52 unless the temperature increase due to resistive heating is limited.

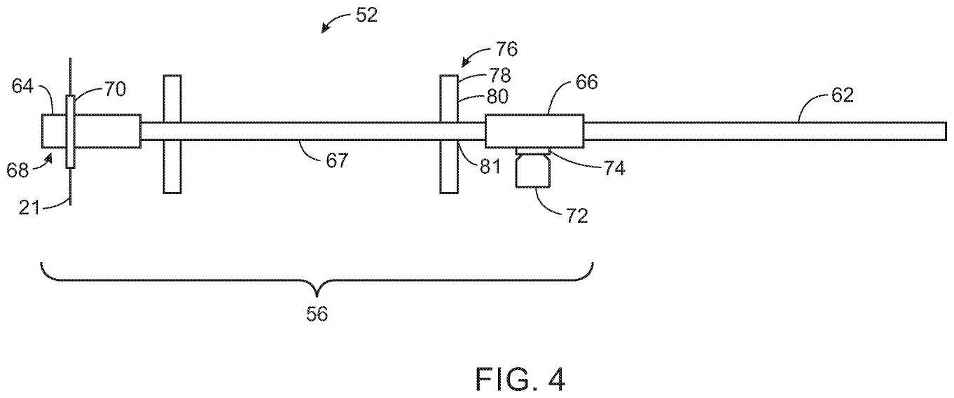

FIG. 4 is a schematic diagram of the current ramp lead 52 (e.g., having a thermal storage device) for utilization with a superconducting magnet of the magnetic resonance system in FIG. 1. The current ramp lead 52 is as described in FIG. 3. The fixed portion of ramp lead assembly 56 includes a thermal storage device 76 (e.g., thermal battery) disposed between the first end 64 and the second end 66 and coupled to the metal rod 67. The thermal storage device 76 is configured to store heat during energization of the superconducting magnet, to limit heat transfer along the metal rod 67 to the second end 66 during the energization of the superconducting magnet, and to limit an increase in temperature along the fixed ramp lead assembly 56 during the energization of the superconducting magnet. The thermal storage device 76 is configured to limit the heat flowing into the thermal anchor 72 during the energization of the superconducting magnet. In addition, the fixed ramp lead assembly 56 is configured so that the heat during the energization of the superconducting machine preferentially flows to the thermal storage device 76 instead of along the metal rod 67 toward the second end 66.

As depicted, thermal storage device 76 includes a solid structure 78 having a suitable solid material (e.g., aluminum, brass, copper, etc.) with a high heat capacity (e.g., material with heat capacities greater than 250 Joules per kilogram per Kelvin (J/kg-K) at 100 K temperature) that limits the temperature increase by absorbing the heat. In certain embodiments, the thermal storage device 76 includes a cryogen tank thermally coupled to the metal rod 67. As depicted, the solid structure 78 is a disc 80 (e.g., metal disc) having a bore 81 that the metal rod 67 passes through. The disc 80 is disposed about the metal rod 67. The configuration of the discs 80 may vary as described in greater detail below. The thermal storage device 76 may include one or more of the discs 80. As depicted, the thermal storage device 76 includes a plurality of the discs 80. Preferably, the discs 80 are made of copper or brass.

The thermal energy created by the resistive heating during the ramping process preferably flows into the discs 80, rather than flow along the higher thermal resistance brass lead material of the metal rod 67. The smaller cross-section, longer brass material of the metal rod 67 that connects the metal ends 64, 66 between the ambient end (e.g., adjacent the first end 64) and the high temperature superconducting power lead 62 has lower thermal conductivity, which reduces the heat transfer along the brass portion. Therefore, the heat transfer is reduced when the superconducting magnet is in operation. Thermal energy flowing into the discs 80 limits the temperature rise and limits the heat flow into the thermal anchor 72 during the ramping process. The number of discs 80, the size of the discs 80, and the location (along the metal rod 67 between the first end 64 and the second end 66) can be optimized.

FIGS. 5-7 are different views of the fixed portion of ramp lead assembly 56 for a pair of current ramp leads (e.g., with the high temperature superconducting power leads of the pair of current ramp leads not shown). The fixed ramp lead assembly 56 includes a support structure 84 (e.g., made of carbon steel). The configuration of the support structure 84 may vary. As depicted, the support structure 84 includes a wall 86 (e.g., vertical wall). The support structure 82 also includes a first pair of rails 88 (e.g., bottom support rails) that are coupled to and extend away from a bottom portion 90 of the wall 86. The support structure 82 further includes a second pair of rails 92 (e.g., top support rails) that are coupled to and extend away from a top portion 94 of the wall 86. The second pair of rails 92 extend at an angle towards ends 96 (located away from the wall 86) of the first pair of rails 88 where they are coupled.

The fixed ramp lead assembly 56 also includes a pair of non-conductive supports 98, 100 coupled to and supported by the first pair of rails 88. Both the first pair of rails 88 and the second pair of rails 92 flank the non-conductive supports 98, 100. The non-conductive supports 98, 100 are spaced apart from each other along the first pair of rails 88. The non-conductive support 98 is coupled to the first pair of rails 88 adjacent to the wall 86. The non-conductive support 100 is coupled to the second pair of rails 92 at the ends 96. The non-conductive supports 98, 100 are configured to provide electrical isolation and high mechanical strength at room temperature. Besides being non-conductive, the non-conductive supports 98, 100 are non-sparking. In certain embodiments, the non-conductive supports 98, 100 are made a thermoset plastic (e.g., glass reinforced epoxy laminate such as G10-FR4).

As depicted a pair of metal rods 67 (e.g., brass rods) are arranged in a parallel manner and coupled to respective first ends 64 (e.g., metal ends made of copper). As noted in FIG. 4, the first ends 64 are coupled to vacuum vessel wall (not shown). Each first end 64 includes a first metal portion 102 (e.g., copper metal portion) extending through both the non-conductive support 98 and the wall 86. Vacuum feedthroughs 106 are provided between the wall 86 and the first metal portions 102. The vacuum feedthroughs 106 are where the fixed ramp lead assembly 56 is coupled to the vacuum vessel wall (i.e., the vacuum vessel wall is disposed about the vacuum feedthroughs 106). Respective electrical insulators 108 are disposed between the first metal portions 102 and the vacuum feedthroughs 106. Every portion of fixed ramp lead assembly 56 is disposed within the space between the vacuum vessel wall and the thermal shield (e.g., outer thermal shield member 164 in FIG. 1) except for the electrical insulators 108 and the first metal portions 102.

The pair of metal rods 67 are coupled to respective second ends 66 (e.g., metal ends made of copper). As depicted, the second ends 66 are stranded copper cables that extend to the thermal anchor (thermal shield, not shown) . . . . The seconds ends 66 extend from respective sockets 110 that are coupled to the metal rods 67 and extend through the non-conductive support 100.

Each metal rod 67 is associated with a respective thermal storage device 76. Each respective thermal storage device 76 includes one or more solid structures 78 having a suitable solid material (e.g., aluminum, brass, copper, etc.) with a high heat capacity (e.g., material with heat capacities greater than 250 Joules per kilogram per Kelvin (J/kg-K) at 100 K temperature) that limits the temperature increase by absorbing the heat. As depicted, the solid structured are discs 80 (e.g., metal disc) having respective bores that the respective metal rod 67 passes through. The discs 80 are disposed about the metal rod 67. The configuration of the discs 80 may vary as described in greater detail below. The thermal storage device 76 may include one or more of the discs 80. Preferably, the discs 80 are made of copper or brass.

The thermal energy created by the resistive heating during the ramping process preferably flows into the discs 80, rather than flow along the higher thermal resistance brass lead material of the metal rod 67. The smaller cross-section, longer brass material of the metal rod 67 that connects the metal ends 64, 66 between the ambient end (e.g., adjacent the first end 64) and the high temperature superconducting power lead 62 has lower thermal conductivity, which reduces the heat transfer along the brass portion. Therefore, the heat transfer is reduced when the superconducting magnet is in operation. Thermal energy flowing into the discs 80 limits the temperature rise and limits the heat flow into the thermal anchor 72 during the ramping process. The number of discs 80, the size of the discs 80, and the location (along the metal rod 67 between the first end 64 and the second end 66) can be optimized. The “disc” is used as a generic term here, the heat storage devices can be any shape to fit the design needs.

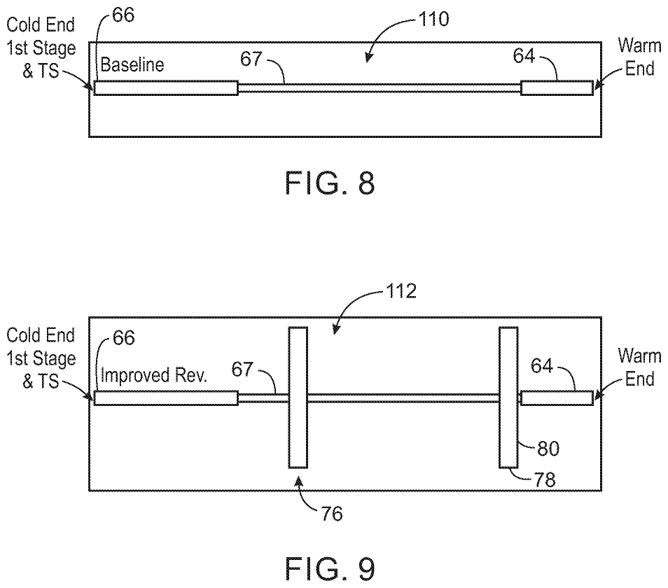

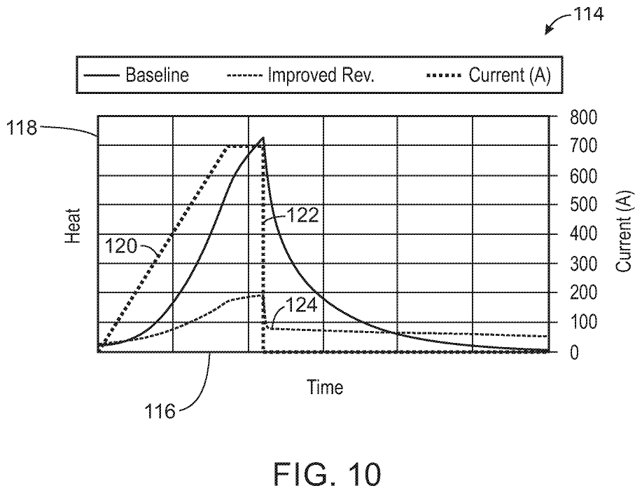

FIG. 10 illustrates the impact of adding the solid structures (e.g., discs) of the thermal storage device to the fixed ramp lead assembly. FIGS. 8 and 9 illustrate the fixed ramp lead assembly utilized in obtaining the simulation data in FIG. 10. FIG. 8 illustrates a fixed ramp assembly 110 (e.g., similar to the fixed ramp assembly 56 in FIG. 3) referred to as the baseline. The fixed ramp assembly 110 includes the metal rod 67 (e.g., brass rod lead) disposed between the first end 64 and the second end 66 (e.g., metal ends such as copper ends). The first end 64 is the warm end (e.g., located adjacent the ambient temperature adjacent vacuum vessel wall). The second end 66 is cold end and is thermally coupled to cold first stage and thermal shield. The fixed ramp assembly 110 lacks the thermal storage device. FIG. 9 illustrates a fixed ramp assembly 112 (e.g., similar to the fixed ramp assembly 56 in FIG. 4) referred to as the improved revision. The fixed ramp assembly 112 includes the metal rod 67 (e.g., brass rod lead) disposed between the first end 64 and the second end 66 (e.g., metal ends such as copper ends). The first end 64 is the warm end (e.g., located adjacent the ambient temperature adjacent vacuum vessel wall). The second end 66 is cold end and is thermally coupled to cold first stage and thermal shield. The fixed ramp assembly 112 includes the thermal storage device 76 in the form of solid structures 78 (e.g., discs 80) as described in FIG. 4.

FIG. 10 is a graph 114 illustrating the heat going into a thermal anchor during a simulated ramping process. The graph 114 includes an X-axis 116 representing time. The graph 114 also includes a Y-axis 118 representing heat into the thermal shield (TS). Plot 120 represents the applied current. Plot 122 represents the baseline fixed ramp assembly 110 in FIG. 8. Plot 124 represents the improved revision of the fixed ramp assembly 112 in FIG. 9 having the thermal storage device. As depicted in FIG. 10, the amount of heat flowing to the thermal shield is reduced significantly when utilizing the fixed ramp assembly 12 having the thermal storage device.

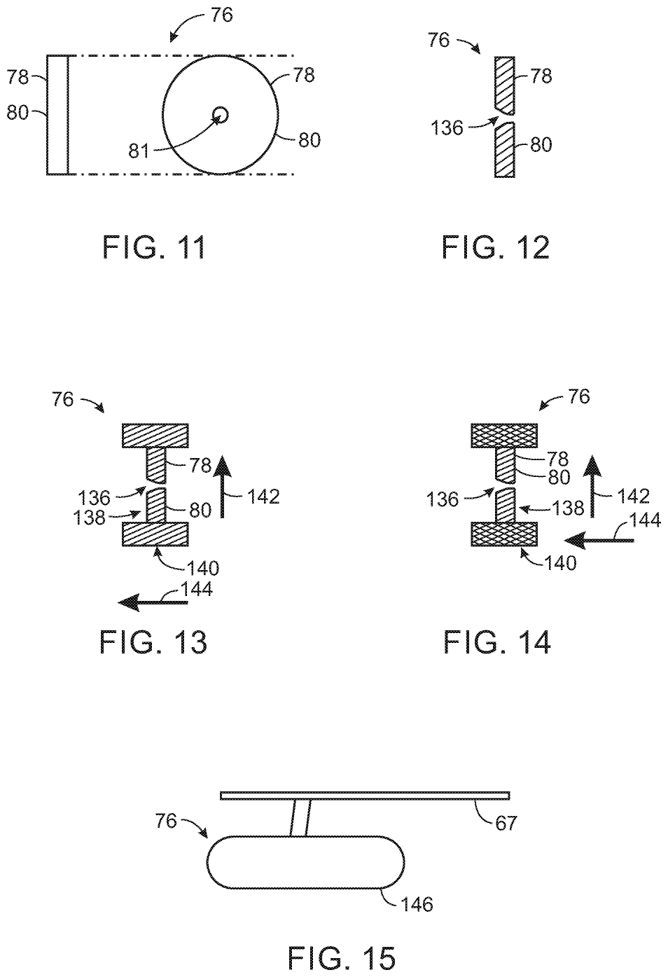

FIGS. 11-15 illustrate different thermal storage devices to be utilized with a fixed ramp lead assembly that serve a temperature limiter. Note that a simple “disc” shape is used for illustrative purposes only, thermal storage devices can have many different shapes to fit design needs. FIG. 11 is an end view and a side view of the thermal storage device 76. The thermal storage device 76 is a solid structure 78 made of a suitable solid material (e.g., metal such as aluminum, brass, copper, etc.) with a high heat capacity (e.g., material with heat capacities greater than 250 Joules per kilogram per Kelvin (J/kg-K) at 100 K temperature) that limits the temperature increase by absorbing the heat. The solid structure 78 is a disc 80 having a bore 81 (e.g., for the metal rod to pass through).

FIG. 12 is an end view of the thermal storage device 76. The thermal storage device 76 is a solid structure 78 made of a suitable solid material (e.g., metal such as aluminum, brass, copper, etc.) with a high heat capacity (e.g., material with heat capacities greater than 250 Joules per kilogram per Kelvin (J/kg-K) at 100 K temperature) that limits the temperature increase by absorbing the heat. The solid structure 78 is a disc 80 having a tapered bore 136 (e.g., for the metal rod to pass through). The tapered bore 136 is configured to reduce axial conduction along the metal rod.

FIG. 13 is an end view of the thermal storage device 76. The thermal storage device 76 is a solid structure 78 made of a suitable solid material (e.g., metal such as aluminum, brass, copper, etc.) with a high heat capacity (e.g., material with heat capacities greater than 250 Joules per kilogram per Kelvin (J/kg-K) at 100 K temperature) that limits the temperature increase by absorbing the heat. The solid structure 78 is a disc 80 having a tapered bore 136 (e.g., for the metal rod to pass through). The tapered bore 136 is configured to reduce axial conduction along the metal rod. The disc 80 includes a central portion 138 having the tapered bore 136. The disc 80 also includes an additional portion 140 disposed along a perimeter of the central portion 138, wherein the central portion 138 extends in a first direction 142 (e.g., vertical direction) and the additional portion 140 extends in a second direction 144 (e.g., horizontal direction) crosswise to the first direction 142. The additional portion 140 is configured to increase a capacity of the disc 80 to store the heat compared to a metal disc only having the central portion 138 (e.g., disc 80 in FIG. 12).

FIG. 14 is an end view of the thermal storage device 76. The thermal storage device 76 is a solid structure 78 made of a suitable solid material (e.g., metal such as aluminum, brass, copper, etc.) with a high heat capacity (e.g., material with heat capacities greater than 250 Joules per kilogram per Kelvin (J/kg-K) at 100 K temperature) that limits the temperature increase by absorbing the heat. The solid structure 78 is a disc 80 having a tapered bore 136 (e.g., for the metal rod to pass through). The tapered bore 136 is configured to reduce axial conduction along the metal rod. The disc 80 includes a central portion 138 having the tapered bore 136. The disc 80 also includes an additional portion 140 disposed along a perimeter of the central portion 138, wherein the central portion 138 extends in a first direction 142 (e.g., vertical direction) and the additional portion 140 extends in a second direction 144 (e.g., horizontal direction) crosswise to the first direction 142. The additional portion 140 is configured to increase a capacity of the disc 80 to store the heat compared to a metal disc only having the central portion 138 (e.g., disc 80 in FIG. 12). The central portion 138 is made of a first metal, and the additional portion 140 is made of second metal different from the first metal. For example, the central portion may be made of copper and the additional portion 140 made of brass, or vice versa.

FIG. 15 is schematic view of the thermal storage device 76. The thermal storage device 76 includes a cryogen tank 146 thermally coupled to metal rod 67 of the fixed ramp lead assembly. The cryogen tank 146 is configured to absorb the thermal energy from the metal rod 67.

FIG. 16 is a schematic diagram of the current or power ramp lead 52 coupled to the superconducting switch 54 coupled to a superconducting electrical machine 148. The current or power ramp lead is as described above and interacts with the superconducting switch as described above (e.g., in FIGS. 4-7). The current or power ramp lead 52 also includes the fixed ramp assembly 56 and the thermal storage device 76 as described above (e.g., in FIGS. 4-7). The current or power ramp lead 52 may be utilized with a superconducting electrical machine 148. The superconducting electrical machine 148 may be a superconducting magnet for magnetic resonance imaging as described above. In certain embodiments, the superconducting electrical machine 148 may be a superconducting motor or a superconducting generator (e.g., for a wind turbine). These are non-limiting examples of superconducting electrical machines 148 that the current or power ramp lead described herein may be utilized with.

Technical effects of the disclosed subject matter include providing for a ramp lead assembly that can be installed on a low cryogen superconducting electrical machine (e.g., superconducting magnet) that provides an always connected electrical path between an external power supply and an internal circuit of the superconducting electrical machine. Thus, the disclosed subject matter is suitable for automatic or remote ramping-up/ramping down of low cryogen sealed superconducting electrical machines that have very limited cryogen (e.g., a few liters of helium). Technical effects of the disclosed subject matter include enabling the automatic ramping up/down of a superconducting magnet in case of a power outage or a natural disaster. Technical effects of the subject matter include increasing superconducting magnet availability for scanning by avoiding a quench for low cryogen (as the recovery from a quench can take several days). Technical effects of the disclosed subject matter include enabling the reduction of heat transfer during operation of the superconducting electrical machine. Technical effects of the disclosed subject matter includes providing for the flow of thermal energy into a thermal storage device to limit a temperature rise and to limit heat flowing into a thermal anchor during a ramping process. The disclosed subject matter includes providing for a simple, low cost, and robust configuration for ramp leads that used for energizing superconducting electrical machines.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A superconducting machine system, comprising:
a superconducting electrical machine;
a cryogenic vessel encompassing the superconducting electrical machine;
a vacuum vessel wall encompassing the cryogenic vessel;
a superconducting switch coupled to the superconducting electrical machine and configured to switch between a resistive mode and a superconducting mode; and
a ramp lead assembly disposed within the vacuum vessel wall and having a first end and a second end, wherein the first end of the ramp lead assembly is coupled in a fixed manner to the vacuum vessel wall and the second end is coupled to a high temperature superconductor power lead coupled to the superconducting switch, wherein the ramp lead assembly comprises:
a non-conductive support;
a metal rod configured to minimize static heat load, wherein the metal rod is disposed on the non-conductive support and extends between the first end and the second end; and
a thermal storage device coupled to the metal rod between the first end and the second end, wherein the thermal storage device is configured to store heat during energization of the superconducting electrical machine, to limit heat transfer along the metal rod to the second end during the energization of the superconducting electrical machine, and to limit an increase in temperature along the ramp lead assembly during the energization of the superconducting electrical machine, wherein a high temperature superconductor portion of the high temperature superconductor power lead is disposed between the second end of metal rod and the superconducting switch.

2. The superconducting machine system of claim 1, wherein the first end and the second end both comprise copper, and the metal rod comprises brass.

3. The superconducting machine system of claim 2, wherein the second end is coupled to a thermal anchor.

4. The superconducting machine system of claim 3, wherein the thermal storage device is configured to limit heat flowing into the thermal anchor during the energization of the superconducting electrical machine.

5. The superconducting machine system of claim 1, wherein a portion of the first end is located outside the vacuum vessel wall, wherein a temperature outside the vacuum vessel wall is an ambient temperature.

6. The superconducting machine system of claim 1, wherein the ramp lead assembly is configured so that heat, during the energization of the superconducting electrical machine, preferentially flows to the thermal storage device instead of along the metal rod toward the second end.

7. The superconducting machine system of claim 1, wherein the thermal storage device comprises a cryogen tank coupled to the metal rod.

8. The superconducting machine system of claim 1, wherein the thermal storage device comprises metal.

9. The superconducting machine system of claim 8, wherein the thermal storage device comprises a metal disc disposed about the metal rod.

10. The superconducting machine system of claim 9, wherein the metal disc comprises brass or copper.

11. The superconducting machine system of claim 9, wherein the thermal storage device comprises a plurality of metal discs disposed about the metal rod spaced apart from each other.

12. The superconducting machine system of claim 9, wherein the metal disc comprises a tapered bore that the metal rod passes through, wherein the tapered bore is configured to reduce axial conduction along the metal rod.

13. The superconducting machine system of claim 12, wherein the metal disc comprises a central portion having the tapered bore and an additional portion disposed along a perimeter of the central portion, wherein the central portion extends in a first direction and the additional portion extends in a second direction crosswise to the first direction, and the additional portion is configured to increase a capacity of the metal disc to store heat compared to another metal disc only having the central portion.

14. The superconducting machine system of claim 13, wherein the central portion is made of a first metal, and the additional portion is made of second metal different from the first metal.

15. A superconducting magnet system for a magnetic resonance imaging system, comprising:
a superconducting magnet;
a cryogenic vessel encompassing the superconducting magnet;
a vacuum vessel wall encompassing the cryogenic vessel;
a superconducting switch coupled to the superconducting magnet and configured to switch between a resistive mode and a superconducting mode; and
a ramp lead assembly disposed within the vacuum vessel wall and having a first end and a second end, wherein the first end of the ramp lead assembly is coupled in a fixed manner to the vacuum vessel wall and the second end is coupled to a high temperature superconductor power lead coupled to the superconducting switch, and wherein the ramp lead assembly comprises:
a non-conductive support;
a metal rod configured to minimize static heat load, wherein the metal rod is disposed on the non-conductive support and extends between the first end and the second end; and
a solid metal structure coupled to the metal rod between the first end and the second end, wherein the ramp lead assembly is configured so that heat, during energization of the superconducting magnet, preferentially flows to the solid metal structure instead of along the metal rod toward the second end, wherein a high temperature superconductor portion of the high temperature superconductor power lead is disposed between the second end of metal rod and the superconducting switch.

16. The superconducting magnet system of claim 15, wherein the second end is coupled to a thermal anchor, and the solid metal structure is configured to store heat during energization of the superconducting magnet, to limit heat transfer along the metal rod to the second end during the energization of the superconducting magnet, to limit an increase in temperature along the ramp lead assembly during the energization of the superconducting magnet, and to limit heat flowing into the thermal anchor during the energization of the superconducting magnet.

17. The superconducting magnet system of claim 15, wherein the solid metal structure comprises a disc.

18. The superconducting magnet system of claim 17, wherein the disc comprises a tapered bore that the metal rod passes through, wherein the tapered bore is configured to reduce axial conduction along the metal rod.

19. The superconducting magnet system of claim 18, wherein the disc comprises a central portion having the tapered bore and an additional portion disposed along a perimeter of the central portion, wherein the central portion extends in a first direction and the additional portion extends in a second direction crosswise to the first direction, and the additional portion is configured to increase a capacity of the disc to store heat compared to a disc only having the central portion.

20. A method for limiting temperature of a ramp lead assembly coupled to a superconducting magnet for a magnetic resonance imaging system, comprising:
energizing the superconducting magnet disposed within a cryogenic vessel disposed within a vacuum vessel wall, wherein the magnetic resonance imaging system comprises:
a ramp lead assembly disposed within the vacuum vessel wall and having a first end and a second end, wherein the first end of the ramp lead assembly is coupled in a fixed manner to the vacuum vessel wall and the second end is coupled to a high temperature superconductor power lead coupled to a superconducting switch, and the ramp lead assembly comprises:
a non-conductive support;
a metal rod that minimize static heat load during energization of the superconducting magnet, wherein the metal rod is disposed on the non-conductive support and extends between the first end and the second end, and wherein a high temperature superconductor portion of the high temperature superconductor power lead is disposed between the second end of the metal rod and the superconducting switch that is configured to further reduce heat transfer into the superconducting magnet; and
a thermal storage device coupled to the metal rod between the first end and the second end; and
storing heat during energization of the superconducting magnet, limiting heat transfer along the metal rod to the second end during the energization of the superconducting magnet, and limiting an increase in temperature along the ramp lead assembly during the energization of the superconducting magnet.
